(19) United States
(12) Patent Application Publication
(10) Pub. No.: US 2004/0153036 A1
(43) Pub. Date: Aug. 5, 2004

(54) Fluid injecting/sampling apparatus having improved barrel and rotary chuck

(75) Inventor: Tzu-Sheng Fan, Miao Li Hsien (TW)

Correspondence Address: BACON & THOMAS, PLLC, 625 SLATERS LANE, FOURTH FLOOR, ALEXANDRIA, VA, 22314

(73) Assignee: TAIJECT MEDICAL DEVICE CO., LTD., HSIN CHU HSIEN (TW)

(21) Appl. No.: 10/755,370

(22) Filed: Jan. 13, 2004

(30) Foreign Application Priority Data
Jan. 17, 2003 (TW) 92200860

(51) Int. Cl. A61M 5/32

(52) U.S. Cl. 604/197

(57) ABSTRACT

A fluid injecting/sampling apparatus is constructed to include a barrel, a needle holder holding a needle cannula, a plunger mounted with a rubber stopper and axially movably mounted in the barrel, and a rotary chuck coupled to the front end of the barrel and prohibited from a reversed rotation after having been turned relative to the barrel to unlock the needle holder for enabling the needle holder to be pulled backwards into the inside of the barrel by the plunger.

### BACKGROUND OF THE INVENTION

1. Field of the Invention

The present invention relates generally to medical implements and, more specifically, to a fluid injecting/sampling apparatus having improved barrel and rotary chuck.

2. Description of the Related Art

There is known a safety hypodermic syringe using a rotary chuck to control the engaging operation of pawls relative to the needle holder. The rotary chuck can be rotated relative to the barrel in reversed directions to lock or unlock the needle holder. Because the rotary chuck is reversible, this design cannot prohibit a reuse of the hypodermic syringe.

Therefore, it is desirable to provide a fluid injecting/sampling apparatus that eliminates the aforesaid drawbacks.

### SUMMARY OF THE INVENTION

The present invention has been accomplished under the circumstances in view. It is the main object of the present invention to provide a fluid injecting/sampling apparatus, which can easily operated to lock/unlock the needle holder. It is another object of the present invention to provide a fluid injecting/sampling apparatus, which prevents a reuse of the apparatus after its service.

To achieve these and other objects of the present invention, the fluid injecting/sampling apparatus comprises a barrel, the barrel having a cylindrical body defining a longitudinally extended receiving hole, an elastically bendable spring strip and an extension portion forwardly extended from a front end of the cylindrical body, a pawl respectively formed on the spring strip at an inner side, an end stop formed on a front end of each the extension portion, a first locating hole and a second locating hole formed in the end stop, and a stop block backwardly axially extended from the end stop; a rotary chuck coupled to the extension portion of the barrel and rotatable relative to the barrel, the rotary chuck having a tubular body defining an axially extended through hole, an escape hole formed in the tubular body, an engagement block disposed inside the tubular body at a front end of the axially extended through hole and respectively engaged into the first locating hole of the barrel, a stop block respectively disposed adjacent to the engagement block and adapted to stop against the stop block of the barrel; and a needle holder, said needle holder having a holder body adapted to hold a needle cannula, and a locating groove extended around the periphery of the holder body and adapted to receive the pawl of the barrel for enabling the needle holder to be locked to the barrel. The rotary chuck is rotatable to move the engagement block over the end stop into engagement with the second locating hole, keeping the stop block stopped at the stop block of the barrel and the escape hole respectively aimed at the spring strip and the pawl.

### BRIEF DESCRIPTION OF THE DRAWINGS

FIG. 1 is an exploded view of a fluid injecting/sampling apparatus having improved barrel and rotary chuck according to one embodiment of the present invention.

FIG. 2 is an enlarged view of a part of FIG. 1, showing the structure of the front part of the barrel and the rotary chuck.

FIG. 3 is an exploded plain view of the barrel and the rotary chuck for the fluid injecting/sampling apparatus according to the present invention.

FIG. 4 is an end view of the rotary chuck for the fluid injecting/sampling apparatus according to the present invention.

FIG. 5 is an assembly view of a part of the fluid injecting/sampling apparatus according to the present invention, showing the rotary chuck coupled to the barrel.

FIG. 6 is a side view in section of the fluid injecting/sampling apparatus according to the present invention, showing the spring strips of the barrel stopped in shape.

FIG. 7 is an end view of FIG. 6.

FIG. 8 is a sectional view taken along line 8-8 of FIG. 6.

FIG. 9 is similar to FIG. 6 but showing the deformable status of the spring strips.

FIG. 10 is an end view of FIG. 9.

FIG. 11 is a sectional view taken along line 11-11 of FIG. 9.

FIG. 12 is a side view in section showing the plunger moved to the rear side of the barrel, the needle holder with the needle cannula received inside the barrel.

### DETAILED DESCRIPTION OF THE INVENTION

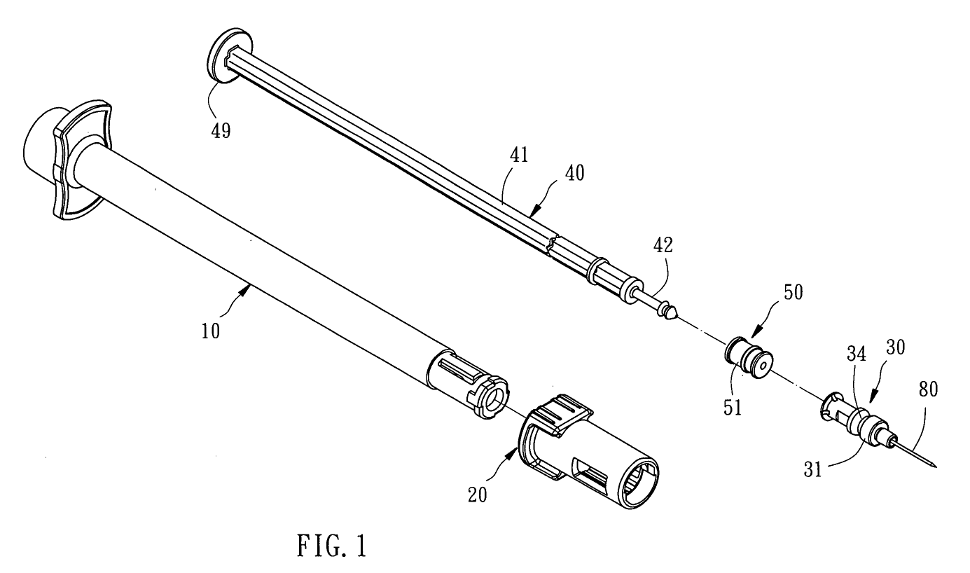

Referring to FIGS. 1˜4, a fluid injecting/sampling apparatus having improved barrel and rotary chuck according to one embodiment of the present invention is shown used with a needle cannula 80. The fluid injecting/sampling apparatus comprises a barrel 10, a rotary chuck 20, a needle holder 30, a plunger 40, and a rubber stopper 50.

The barrel 10 has a cylindrical body 11 defining a longitudinally extended receiving hole 12, two elastically bendable opposing spring strips 13 and two opposing extension portions 15 forwardly extended from the front end of the cylindrical body 11, two pawls 14 respectively formed on the free ends of the spring strips 13 and facing each other, an end stop 16 formed on the front ends of the extension portions 15, two L-shaped first locating holes 17 and two L-shaped second locating holes 19 alternatively and equiangularly spaced around the periphery of the end stop 16, and two opposing stop rods 18 backwardly axially extended from the end stop 18.

The rotary chuck 20 has a tubular body 21 defining an axially extended through hole 22, two escape holes 23 formed in the peripheral wall of the tubular body 21 at two sides, two finger strips 24 bilaterally formed on the peripheral wall of the tubular body 21 at the rear end, a plurality of ribs 25 formed on the inside of the through hole 22, two engagement blocks 27 bilaterally disposed in the front end of the through hole 22 and adapted to engage the first locating holes 17 of the barrel 10, two stop blocks 28 respectively disposed adjacent to the inner side of the engagement blocks 27 and adapted to stop against the stop rods 18 of the barrel 10.

The needle holder 30 has a holder body 31 adapted to hold a needle cannula 80, and a locating groove 34 extended around the periphery of the holder body 31 and adapted to receive the pawls 14 of the barrel 10.

The plunger 40 has an elongated plunger body 41, a stopper retainer 42 disposed at the front end of the plunger body 41, and a thumb rest 49 provided at the rear end of the plunger body 41 for the rest of the thumb.

The rubber stopper 50 has a stopper body 51 fastened to the stopper retainer 42 and supported on the front end of the plunger body 41 and peripherally disposed in close contact with the inside wall (the peripheral wall of the receiving hole 12) of the inner barrel 10.

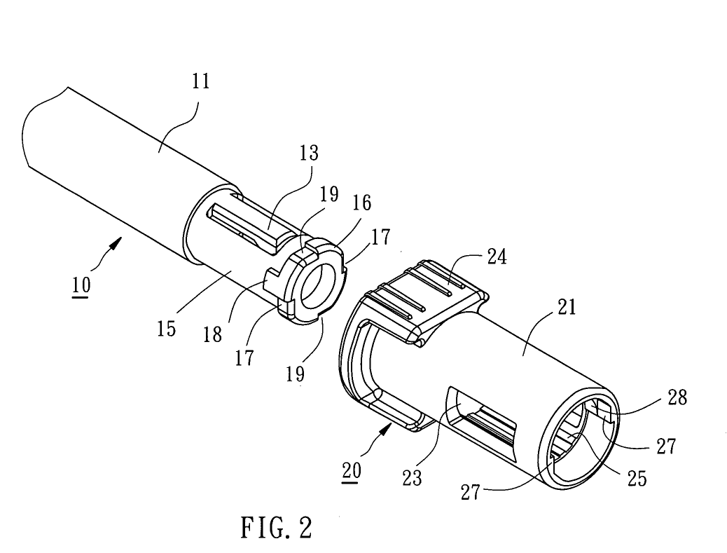

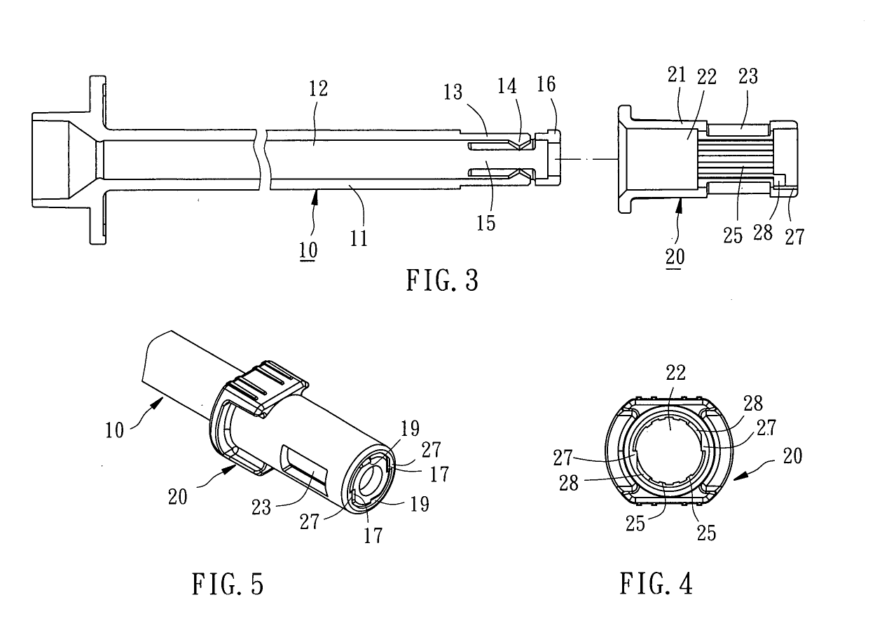

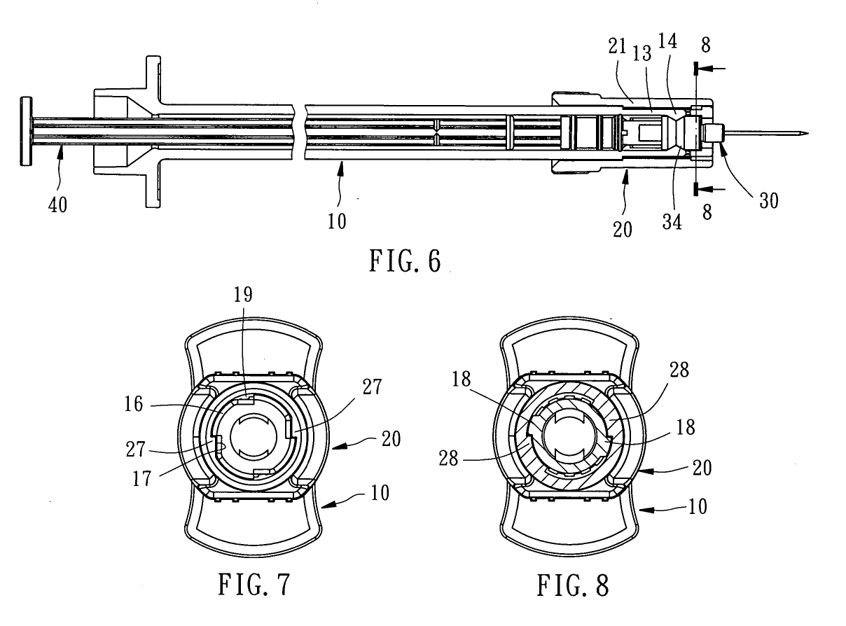

Referring to FIGS. 2 and 3, by means of the through hole 22, the rotary chuck 20 is sleeved onto the end stop 16 of the barrel 10 for enabling the ribs 25 of the rotary chuck 20 to be stopped at the extension portions 15 of the barrel 10. When sleeving the rotary chuck 20 onto the end stop 16 of the barrel 10, the stop blocks 28 are forced to deform and to pass over the end stop 16. When the stop blocks 28 moved over the end stop 16, the rotary chuck 20 is prohibited from backward movement relative to the barrel 10, i.e., the end stop 16 stops the rotary chuck 20 from falling out of the barrel 10. When installed, the engagement blocks 27 are respectively received in the first locating holes 17.

FIGS. 5˜8 show the status of the fluid injecting/sampling apparatus before use where the needle holder 30 is locked. Before rotating the rotary chuck 20 relative to the barrel 10, the tubular body 21 of the rotary chuck 20 is disposed around the spring strips 13 to hold the spring strips 13 in shape, i.e., the pawls 14 of the barrel 10 are held in engagement with the locating groove 34 of the needle holder 30, and therefore the needle holder 30 is locked to the barrel 10 for normal operation.

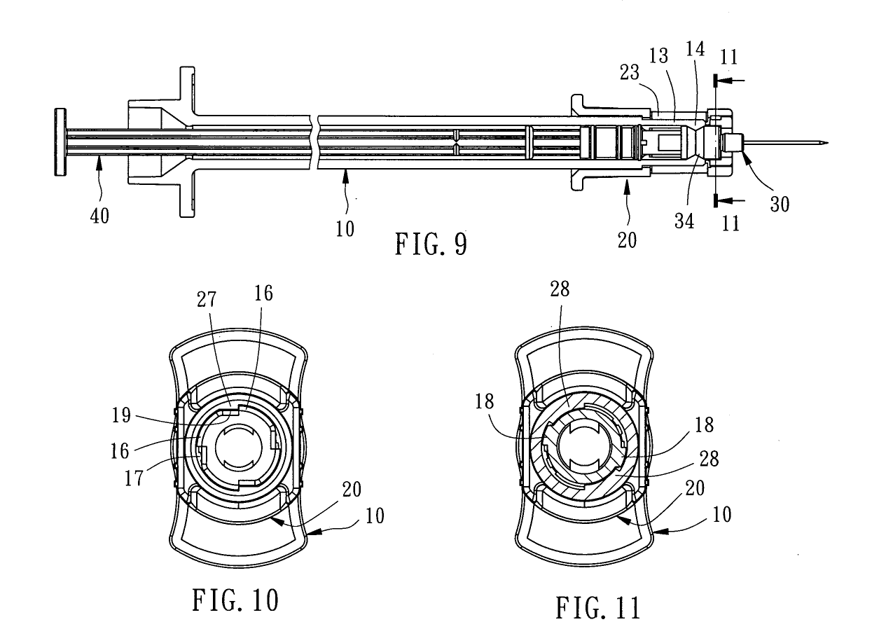

FIGS. 9˜11 show the needle holder 30 unlocking operation. When rotated the rotary chuck 20 relative to the barrel 10 through an angle to move the engagement blocks 27 over the end stop 16 of the barrel 10, the escape holes 23 of the rotary chuck 20 are moved to the periphery of the spring strips 13 for enabling the spring strips 13 to be curved outwards, i.e., when pulling the plunger 40 backwards, the pawls 14 of the barrel 10 will be disengaged from the engagement blocks 27 for enabling the needle holder 30 to be pulled backwards with the plunger 40 to the inside of the barrel 10.

At this time, as shown in FIG. 10, the engagement blocks 27 are stopped by the end stop 16 of the barrel 10 to prohibit the rotary chuck 20 from reversed rotation, therefore the needle holder 30 cannot be locked to the barrel 10 for normal use again, i.e., the fluid injecting/sampling apparatus cannot be used again.

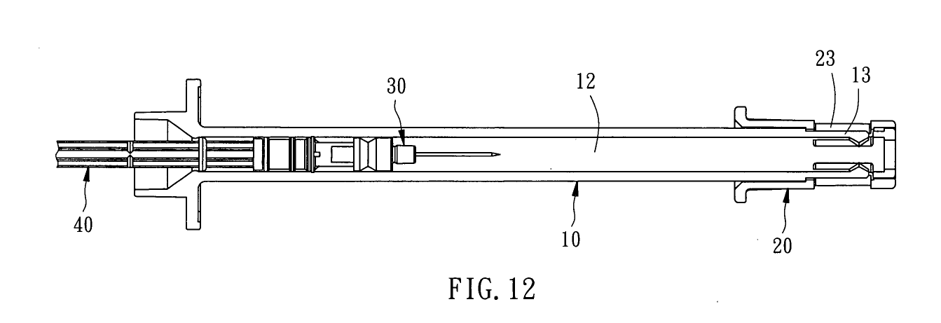

FIG. 12 shows the needle holder 30 moved backwards with the plunger 40 and received inside the receiving hole 12 of the barrel 10, keeping the needle cannula 80 out of touch of a person.

As indicated above, the fluid injecting/sampling apparatus of the present invention has the following features:

1. The rotary chuck and the barrel of the present invention can easily be assembled, and the rotary chuck can easily be operated to control engagement of the pawls with the needle holder.

2. After rotary motion of the rotary chuck to unlock the needle holder from the barrel, the rotary chuck is prohibited from reversed rotation, and the pawls of the barrel cannot be forced into engagement with the needle holder again, prohibiting a reuse of the fluid injecting/sampling apparatus.

What is claimed is:

1. A fluid injecting/sampling apparatus comprising:
a barrel, said barrel having a cylindrical body defining a longitudinally extended receiving hole, an elastically bendable spring strip and an extension portion forwardly extended from a front end of said cylindrical body, a pawl respectively formed on said spring strip at an inner side, an end stop formed on a front end of each said extension portion, a first locating hole and a second locating hole formed in said end stop, and a stop block backwardly axially extended from said end stop;
a rotary chuck coupled to said extension portion of said barrel and rotatable relative to said barrel, said rotary chuck having a tubular body defining an axially extended through hole, an escape hole formed in said tubular body, an engagement block disposed inside said tubular body at a front end of said axially extended through hole and respectively engaged into said first locating hole of said barrel, a stop block respectively disposed adjacent to said engagement block and adapted to stop against said stop block of said barrel; and
a needle holder, said needle holder having a holder body adapted to hold a needle cannula, and a locating groove extended around the periphery of said holder body and adapted to receive said pawl of said barrel for enabling said needle holder to be locked to said barrel;
wherein said rotary chuck is rotatable to move said engagement block over said end stop into engagement with said second locating hole, keeping said stop block stopped at said stop block of said barrel and said escape hole respectively aimed at said spring strip and said pawl.

2. The fluid injecting/sampling apparatus as claimed in claim 1, wherein the number of said spring strip of said barrel is two, and the number of said escape hole of said rotary chuck is two.

3. The fluid injecting/sampling apparatus as claimed in claim 1, wherein the number of said first locating hole and the number of said second locating hole of said barrel are two, and the number of said engagement block of said rotary chuck is two.

4. The fluid injecting/sampling apparatus as claimed in claim 1, wherein said locating hole of said barrel each has an L-shaped profile.

5. The fluid injecting/sampling apparatus as claimed in claim 1, wherein the number of said stop block of said barrel is two.

6. The fluid injecting/sampling apparatus as claimed in claim 1, wherein the number of said stop block of said rotary chuck is two.

7. The fluid injecting/sampling apparatus as claimed in claim 1, wherein said rotary chuck has a plurality of ribs formed on the inside of said axially extended through hole.

8. The fluid injecting/sampling apparatus as claimed in claim 1, further comprising a plunger adapted to move said needle holder.

9. The fluid injecting/sampling apparatus as claimed in claim 8, further comprising a rubber stopper fastened to a front end of said plunger.

10. The fluid injecting/sampling apparatus as claimed in claim 1, wherein said stop block of said rotary chuck is respectively disposed adjacent to an inner side of each said engagement block.
